(19) United States
(12) Patent Application Publication
(10) Pub. No.: US 2025/0268803 A1
(43) Pub. Date: Aug. 28, 2025

(54) SUNSCREEN PREPARATION

(71) Applicant: BEIERSDORF AG, Hamburg (DE)

(72) Inventors: Yongyi ZHANG, New Providence, NJ (US); Donald RICK, Dumont, NJ (US)

(21) Appl. No.: 18/585,371

(22) Filed: Feb. 23, 2024

(51) Int. Cl. A61K 8/04; A61Q 17/04

(52) U.S. Cl. CPC A61K 8/042; A61Q 17/04; A61K 2800/31; A61K 2800/5922

(57) ABSTRACT

A sunscreen preparation for topical application to skin comprises one or more organic UV filter substances as well as Triheptanoin, Coco-Caprylate/Caprate, Dilinoleic Acid/Butanediol Copolymer, Castor Oil/IPDI Copolymer, Poly C10-30 Alkyl Acrylate, Hydroxystearic Acid, and Dextrin Palmitate.

### BACKGROUND OF THE INVENTION

### 1. Field of the Invention

The present invention relates to a sunscreen preparation for topical application to skin which comprises one or more UV filter substances and several components which allow the preparation to be translucent even in the absence of a silicone elastomer.

### 2. Discussion of Background Information

There is a trend that consumers in North America are looking for sunscreen options made specially for their skin tones to avoid a white cast after applying sunscreen lotions. Sunscreen lotions tinted with iron oxide are an option to reduce the white cast on darker skin tone. However, the use level of the pigment needs to be adjusted for each different skin tone. Translucent sunscreens, in contrast, can be used for all the consumers' skin tones.

There are several translucent sunscreen products available on the US market. A commonly used formulation strategy is to disperse organic UV filter substances into silicone elastomer blends. The resultant anhydrous translucent gel offers silky and smooth skin feel at a high use level of silicone elastomer (40% and more). However, the high use level of silicone elastomer brings the overall cost of the formula up. More importantly, due to compatibility issues between silicone elastomer and organic UV filter substances this type of formulas cannot provide high UV protection, as evidenced by in-vivo and in-vitro SPF testing results.

In view of the foregoing, it would be advantageous to have available a preferably translucent or clear sunscreen preparation for topical application to skin which comprises one or more UV filter substances, provides a high SPF factor and does not require high concentrations of silicone elastomer.

### SUMMARY OF THE INVENTION

The present invention provides a sunscreen preparation for topical application to skin. The preparation comprises one or more organic UV filter substances and
(i) Triheptanoin
(ii) Coco-Caprylate/Caprate
(iii) Dilinoleic Acid/Butanediol Copolymer
(iv) Castor Oil/IPDI Copolymer (IPDI stands Isophorone Diisocyanate)
(v) Poly C10-30 Alkyl Acrylate
(vi) Hydroxystearic Acid
(vii) Dextrin Palmitate.

In one embodiment, the preparation further comprises
(viii) Dicaprylyl Carbonate
(ix) Stearalkonium Hectorite
(x) Propylene Carbonate
   and/or further comprises
(x) Propylene Carbonate
(xi) Dimethicone
(xii) Ethylhexyl palmitate
(xiii) Quaternium-90 Bentonite.

In another embodiment, the preparation is substantially free of silicone elastomer and/or is substantially free of water.

In another embodiment, the preparation is clear or translucent and/or is present as a gel or lotion.

In another embodiment, the preparation comprises the one or more organic UV filter substances in a total concentration of from about 2% to about 40% by weight, based on the total weight of the preparation. By way of example, the one or more organic UV filter substances may comprise one or more of butyl methoxydibenzoylmethane, homosalate, octocrylene, and octisalate.

In another embodiment, the preparation comprises components (i), (ii), (iii) and (iv) in a total concentration of from 1% to 10% by weight and/or comprises components (v), (vi) and (vii) in a total concentration of from 1% to 10% by weight, each based on the total weight of the preparation.

In another embodiment, the preparation comprises components (i), (ii), (iii), (iv) (v), (vi), and (vii) in a total concentration of from 2% to 20% by weight, based on the total weight of the preparation.

In another embodiment, the preparation comprises components (viii), (ix) and (x) in a total concentration of from 1% to 10% by weight and/or comprises components (x), (xi), (xii) and (xiii) in a total concentration of from 1% to 10% by weight, each based on the total weight of the preparation.

In another embodiment, the ratio of the total weight of components (i), (ii), (iii) and (iv) to the total weight of components (v), (vi) and (vii) is from 3:1 to 1:3.

In another embodiment, the preparation comprises component (v) and component (vi) in a weight ratio of from 6:1 to 2:1 and/or comprises component (v) and component (vii) in a weight ratio of from 2:1 to 1:2.

In another embodiment, the preparation has an in-vitro SPF of at least 30.

In another embodiment, the preparation further comprises one or more of dibutyl adipate, silica dimethyl silylate, neopentyl glycol diheptanoate, butylene glycol dicaprylate/dicaprate, hydroxyacetophenone, silica.

The present invention further provides a method of protecting skin from UV radiation. The method comprises applying to the skin to be protected the sunscreen preparation as set forth above, including the various embodiments thereof.

As set forth above, the preparation of the present invention comprises one or more organic UV filter substances. Usually the preparation comprises at least two, e.g., at least three or at least four organic UV filter substances. These UV filter substances can be selected from all organic UV filter substances. The one or more organic UV filter substances will often be present in a total concentration of from about 2% to about 40% by weight, based on the total weight of the preparation, although higher and lower concentrations are possible as well. Usually, the one or more organic UV filter substances are present in a total concentration of at least 5%, e.g., at least 10%, at least 15%, at least 20%, at least 25% or at least 30% by weight, based on the total weight of the preparation.

The preparation of the present invention further comprises components (i) to (vii) as set forth above. Components (i), (ii), (iii) and (iv) will often be present in a total concentration of from 1% to 10% by weight, based on the total weight of the preparation. For example, components (i), (ii), (iii) and (iv) may be present in a total concentration of at least 2% by weight, e.g., at least 3% or at least 4% by weight, and not higher than 8%, not higher than 7%, not higher than 6% or not higher than 5% by weight.

Components (v), (vi) and (vii) will often be present in a total concentration of from 1% to 10% by weight, based on the total weight of the preparation. For example, (v), (vi) and (vii) may be present in a total concentration of at least 2% by weight, e.g., at least 3%, at least 4% or at least 5% by weight, and not higher than 8%, not higher than 7% or not higher than 6% by weight.

The total concentration of components (i), (ii), (iii), (iv) (v), (vi) and (vii) in the preparation of the present invention will often be from 2% to 20% by weight, e.g., from 4% to 15% or from 7% to 12% by weight, based on the total weight of the preparation.

The total concentration of components (i), (ii), (iii) and (iv) to the total concentration of components (v), (vi) and (vii) in the preparation will often be from 3:1 to 1:3, e.g., from 2:1 to 1:2, or from 1.5:1 to 1:1.5.

The weight ratio of component (v) to component (vi) will often be from 6:1 to 2:1, e.g., from 5:1 to 3:1, and the weight ratio of component (v) to component (vii) will often be from 2:1 to 1:2, e.g., from 1.5:1 to 1:1.5 or from 1.3:1 to 1:1.3.

A combination of components (i), (ii), (iii) and (iv) is commercially available as Gransense TC-21X/C from Grant Industries, US, (v) is commercially available as, e.g., Tego SP 13-6 from Evonik Personal Care, US. Component (vi) is commercially available as, e.g., CASID HSA from Aston Chemicals Ltd., UK. Component (vii) is commercially available as, e.g., Rheopearl KL2 from Miyoshi Europe, France.

A combination of components (viii), (ix) and (x) is commercially available as, e.g., Cosmedia Gel CC from BASF AG, Germany, and a combination of components (x), (xi), (xii) and (xiii) is commercially available as, e.g., Ggel-100V from Applechem, US.

The preparation of the invention will often have an in-vitro SPF of at least 30, e.g., at least 35 or at least 38, measured on PMMA plates by a Labsphere 2000 UV Transmittance Analyzer with an applied amount of sunscreen sample on the PMMA plate of 0.75 mg/cm².

The preparation of the invention will usually be substantially free of silicone elastomer and substantially free of water.

“Substantially free of silicone elastomer” means that the preparation contains not more than 1%, preferably not more than about 0.5%, more preferably not more than about 0.1% by weight, e.g., not more than about 0.01% by weight, of silicone elastomer(s), based on the total weight of the preparation.

“Substantially free of water” means that the preparation contains not more than about 1%, preferably not more than about 0.5%, more preferably not more than about 0.1% by weight e.g., not more than about 0.01% by weight, of water, based on the total weight of the preparation.

The preparation of the invention preferably is clear or translucent. It may be in any form that is suitable for topical application to human skin but preferably is present as a gel or as a lotion.

In addition to the components set forth above the preparation of the invention will usually contain one or more of components which are commonly employed for making sunscreen preparations, such as, for example, substances selected from organic solvents (e.g., glycerol, alkanols and alkanediols such as e.g., ethanol, ethers, esters, etc.), inorganic and organic thickeners, natural and synthetic oils and waxes, substances for enhancing the skin feel and/or the spreadability of the preparation, substances for soothing the skin, antioxidants, moisturizers, emollients, active ingredients, substances for enhancing the water-resistance of the preparation. Non-limiting examples of corresponding substances include dibutyl adipate, silica dimethyl silylate, neopentyl glycol diheptanoate, butylene glycol dicaprylate/dicaprate, hydroxyacetophenone, silica, tocopherol, tocopheryl acetate, hyaluronic acid, and sodium hyaluronate.

To sum up, the present invention provides the following items:

1. A sunscreen preparation for topical application to skin, wherein the preparation comprises one or more organic UV filter substances and
(i) Triheptanoin
(ii) Coco-Caprylate/Caprate
(iii) Dilinoleic Acid/Butanediol Copolymer
(iv) Castor Oil/IPDI Copolymer
(v) Poly C10-30 Alkyl Acrylate
(vi) Hydroxystearic Acid
(vii) Dextrin Palmitate.

2. The sunscreen preparation of item 1, wherein the preparation further comprises
(viii) Dicaprylyl Carbonate
(ix) Stearalkonium Hectorite
(x) Propylene Carbonate.

3. The sunscreen preparation of item 1 or item 2, wherein the preparation further comprises
(x) Propylene Carbonate
(xi) Dimethicone
(xii) Ethylhexyl palmitate
(xiii) Quaternium-90 Bentonite.

4. The sunscreen preparation of any one of the preceding items, wherein the preparation is substantially free of silicone elastomer.

5. The sunscreen preparation of any one of the preceding items, wherein the preparation is substantially free of water.

6. The sunscreen preparation of any one of the preceding items, wherein the preparation is clear or translucent.

7. The sunscreen preparation of any one of the preceding items, wherein the preparation is present in the form of a gel.

8. The sunscreen preparation of any one of the preceding items, wherein the preparation comprises the one or more organic UV filter substances in a total concentration of from about 2% to about 40% by weight, based on a total weight of the preparation.

9. The sunscreen preparation of any one of the preceding items, wherein the one or more organic UV filter substances comprise one or more of butyl methoxydibenzoylmethane, homosalate, octocrylene, and octisalate.

10. The sunscreen preparation of any one of the preceding items, wherein the preparation comprises components (i), (ii), (iii) and (iv) in a total concentration of from 1% to 10% by weight, based on a total weight of the preparation.

11. The sunscreen preparation of any one of the preceding items, wherein the preparation comprises components (v), (vi) and (vii) in a total concentration of from 1% to 10% by weight, based on a total weight of the preparation.

12. The sunscreen preparation of any one of the preceding items, wherein the preparation comprises components (i), (ii), (iii), (iv) (v), (vi) and (vii) in a total concentration of from 2% to 20% by weight, based on a total weight of the preparation.

13. The sunscreen preparation of any one of the preceding items, wherein the preparation comprises components (viii), (ix) and (x) in a total concentration of from 1% to 10% by weight, based on a total weight of the preparation.

14. The sunscreen preparation of any one of the preceding items, wherein the preparation comprises components (x), (xi), (xii) and (xiii) in a total concentration of from 1% to 10% by weight, based on a total weight of the preparation.

15. The sunscreen preparation of any one of the preceding items, wherein the ratio of total weight of components (i), (ii), (iii) and (iv) to total weight of components (v), (vi) and (vii) is from 3:1 to 1:3.

16. The sunscreen preparation of any one of the preceding items, wherein the weight ratio of component (v) to component (vi) is from 6:1 to 2:1.

17. The sunscreen preparation of any one of the preceding items, wherein the weight ratio of component (v) to component (vii) is from 2:1 to 1:2.

18. The sunscreen preparation of any one of the preceding items, wherein the preparation has an in-vitro SPF of at least 30.

19. The sunscreen preparation of any one of the preceding items, wherein the preparation further comprises one or more of dibutyl adipate, silica dimethyl silylate, neopentyl glycol diheptanoate, butylene glycol dicaprylate/dicaprate, hydroxyacetophenone, silica.

20. A method of protecting skin from UV radiation, wherein the method comprises applying to the skin to be protected the sunscreen preparation of any one of the preceding items.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those of skill in the art how the several forms of the present invention may be embodied in practice.

To develop a translucent sunscreen with similar sensorial performance, high UV protection, and lower formula cost the following four key components were used.

### Composition 1: Gransense TC-21X/C

INCI: Triheptanoin (and) Coco-Caprylate/Caprate (and) Dilinoleic Acid/Butanediol Copolymer (and) Castor Oil/IPDI Copolymer

Gransense TC-21X/C is a non-volatile naturally derived, bio-based elastomer. As a silicone elastomer replacement, Gransense TC-21X/C can mimic the skin feel at much lower use level (around 5%). Additionally, the combination of polymers and cosmetic oils in Gransense TC-21X/C enhances compatibility with high polarity organic UV filter substances.

### Compositions 2 and 3: Clay Materials

INCI (Cosmedia Gel CC): Dicaprylyl Carbonate (and) Stearalkonium Hectorite (and) Propylene Carbonate

INCI (Ggel-100V): Dimethicone (and) Ethylhexyl palmitate (and) Quatermium-90 Bentonite (and) Propylene Carbonate

Both clay materials work as cosmetic oil thickeners and sensory modifiers. They provide a smooth and lubricating feel to skin. They also offer shear-thinning viscosity control and oil structuring property.

### Composition 4: Oil Thickeners

INCI (Tego SP 13-6): Poly C10-30 Alkyl Acrylate

INCI (CASID HSA): Hydroxystearic Acid

INCI (Rheopearl KL2): Dextrin Palmitate

The combination of these materials increases the viscosity of the cosmetic oils while ensuring the clarity of a sunscreen gel. The softness and sensory properties of the sunscreen gel can be controlled by tuning the concentrations of these oil thickening materials. These oil thickeners also provide an SPF boosting effect and enhance the water resistance of the preparation. Formulators can control the overall formula cost by tuning the concentration of each material.

### EXAMPLES

Examples of suitable preparations are shown in Table I to compare the UV protection. The preparations of Example 1 and Example 2 are preparations with silicone elastomer at different use levels. The preparations of Example 3 and Example 4 are preparations which combine the silicone elastomer and bio-based elastomer at different use levels. The preparation of Example 5 is a preparation with only bio-based elastomer. The preparations of Examples 2 to 5 contain oil thickeners to control the overall formula viscosity.

The preparation of Example 5 with only bio-based elastomer is more translucent compared to the formulas with large amount of silicone elastomer. The main reason is that the bio-based silicone elastomer is more compatible with the sunscreen filters and high polarity emollients.

TABLE I
Formula Compositions (% by weight)

| INCI Name | Ex. 1 | Ex. 2 | Ex. 3 | Ex. 4 | Ex. 5 |
|---|---|---|---|---|---|
| Dimethicone (&) Dimethicone | 40 | 25 | 8 | 4 | — |
| Crosspolymer | | | | | |
| Triheptanoin (&) Coco- | — | — | 4.5 | 4.5 | 4.5 |
| Caprylate/Caprate (&) Dilinoleic | | | | | |
| Acid/Butanediol Copolymer (&) | | | | | |
| Castor Oil/IPDI Copolymer | | | | | |
| Dibutyl Adipate | 10 | 10 | 10 | 10 | 10 |
| Butyl Methoxydibenzoylmethane | 3 | 3 | 3 | 3 | 3 |
| Homosalate | 15 | 15 | 15 | 15 | 15 |
| Octocrylene | 10 | 10 | 10 | 10 | 10 |
| Octisalate | 5 | 5 | 5 | 5 | 5 |
| Silica Dimethyl Silylate | 5 | 5 | 5 | 5 | 5 |
| Neopentyl Glycol Diheptanoate | 0.7 | 10.2 | 22.7 | 26.7 | 30.7 |
| Butylene Glycol | 10 | 10 | 10 | 10 | 10 |
| Dicaprylate/Dicaprate | | | | | |
| Dextrin Palmitate | — | 3 | 3 | 3 | 3 |
| Poly C10-30 Alkyl Acrylate | — | 2 | 2 | 2 | 2 |
| Hydroxystearic Acid | — | 0.5 | 0.5 | 0.5 | 0.5 |
| Hydroxyacetophenone | 0.3 | 0.3 | 0.3 | 0.3 | 0.3 |
| Silica | 1 | 1 | 1 | 1 | 1 |
| In-vitro SPF | 12.89 | 28.28 | 30.28 | 32.68 | 38.81 |

As shown in Table I, the in-vitro SPF value increases as the percentage of the silicone elastomer decreases. The highest in-vitro SPF value was observed in Example 5 with only bio-based elastomer (with oil thickeners) present in the preparation.

Additional example preparations are listed in Table II below to show the synergistic UV protective effect when combining bio-based elastomer, clay material, and oil thickeners.

The clay material brings a slightly yellow color into the formula. But all the preparations show similar clarity as translucent sunscreen oil gels.

The highest in-vitro SPF value is observed in Example 7. All key ingredients (Bio-based elastomer, Clay material, and Oil thickeners) were used in the preparation of Example 7.

TABLE II
Formula Compositions (% by weight)

| INCI Name | Ex. 6 | Ex. 7 | Ex. 8 | Ex. 9 |
|---|---|---|---|---|
| Dimethicone (&) Dimethicone | — | — | — | 4 |
| Crosspolymer | | | | |
| Triheptanoin (&) Coco- | 4.5 | 4.5 | 4.5 | 4.5 |
| Caprylate/Caprate (&) Dilinoleic | | | | |
| Acid/Butanediol Copolymer (&) | | | | |
| Castor Oil/IPDI Copolymer | | | | |
| Dicaprylyl Carbonate (&) | — | 4 | 7.5 | 7.5 |
| Stearalkonium Hectorite (&) | | | | |
| Propylene Carbonate | | | | |
| Dibutyl Adipate | 10 | 10 | 10 | 10 |
| Butyl Methoxydibenzoylmethane | 3 | 3 | 3 | 3 |
| Homosalate | 15 | 15 | 15 | 15 |
| Octocrylene | 10 | 10 | 10 | 10 |
| Octisalate | 5 | 5 | 5 | 5 |
| Silica Dimethyl Silylate | 5 | 5 | 5 | 5 |
| Neopentyl Glycol Diheptanoate | 30.7 | 26.7 | 23.2 | 19.2 |
| Butylene Glycol | 10 | 10 | 10 | 10 |
| Dicaprylate/Dicaprate | | | | |
| Dextrin Palmitate | 3 | 3 | 3 | 3 |
| Poly C10-30 Alkyl Acrylate | 2 | 2 | 2 | 2 |
| Hydroxystearic Acid | 0.5 | 0.5 | 0.5 | 0.5 |
| Hydroxyacetophenone | 0.3 | 0.3 | 0.3 | 0.3 |
| Silica | 1 | 1 | 1 | 1 |
| In-vitro SPF | 38.81 | 42.70 | 38.56 | 36.78 |

The in-vitro SPF values were measured on PMMA plates by a Labsphere 2000 UV Transmittance Analyzer. The applied amount of sunscreen sample on the PMMA plate was 0.75 mg/cm².

Preparations with similar formulation strategies were evaluated in in-vivo SPF tests. The results obtained are shown in Table III. The preparation of Example 10 with Bio-based elastomer, Clay material, and Oil thickeners resulted in a synergistic UV protection in static and 40 minute water resistance tests. If silicone elastomer is included in the preparation (Example 11), the SPF values (in-vivo static and 40 minutes water resistance) decrease significantly.

TABLE III
In-vivo SPF results/ Formula Compositions (% by weight)

| | Ex. 10 | Ex. 11 |
|---|---|---|
| | (Similar to Ex. 8) | (Similar to Ex. 3) |
| Bio-based elastomer | 4.5 | 4 |
| Silicone elastomer | — | 8 |
| Clay material | 7.5 | 7.5 |
| Oil thickeners | Yes | Yes |
| In-vivo Static SPF (Mean) | 66.2 | 40.6 |
| 40 minutes SPF (Mean) | 59.9 | 44.7 |

What is claimed is:

1. A sunscreen preparation for topical application to skin, wherein the preparation comprises one or more organic UV filter substances and
(i) Triheptanoin
(ii) Coco-Caprylate/Caprate
(iii) Dilinoleic Acid/Butanediol Copolymer
(iv) Castor Oil/IPDI Copolymer
(v) Poly C10-30 Alkyl Acrylate
(vi) Hydroxystearic Acid
(vii) Dextrin Palmitate.

2. The sunscreen preparation of claim 1, wherein the preparation further comprises
(viii) Dicaprylyl Carbonate
(ix) Stearalkonium Hectorite
(x) Propylene Carbonate.

3. The sunscreen preparation of claim 1, wherein the preparation further comprises
(x) Propylene Carbonate
(xi) Dimethicone
(xii) Ethylhexyl palmitate
(xiii) Quaternium-90 Bentonite.

4. The sunscreen preparation of claim 1, wherein the preparation is substantially free of silicone elastomer.

5. The sunscreen preparation of claim 1, wherein the preparation is substantially free of water.

6. The sunscreen preparation of claim 1, wherein the preparation is clear or translucent.

7. The sunscreen preparation of claim 1, wherein the preparation is present in the form of a gel or a lotion.

8. The sunscreen preparation of claim 1, wherein the preparation comprises the one or more organic UV filter substances in a total concentration of from about 2% to about 40% by weight, based on a total weight of the preparation.

9. The sunscreen preparation of claim 1, wherein the one or more organic UV filter substances comprise one or more of butyl methoxydibenzoylmethane, homosalate, octocrylene, and octisalate.

10. The sunscreen preparation of claim 1, wherein the preparation comprises components (i), (ii), (iii) and (iv) in a total concentration of from 1% to 10% by weight, based on a total weight of the preparation.

11. The sunscreen preparation of claim 1, wherein the preparation comprises components (v), (vi) and (vii) in a total concentration of from 1% to 10% by weight, based on a total weight of the preparation.

12. The sunscreen preparation of claim 1, wherein the preparation comprises components (i), (ii), (iii), (iv) (v), (vi) and (vii) in a total concentration of from 2% to 20% by weight, based on a total weight of the preparation.

13. The sunscreen preparation of claim 2, wherein the preparation comprises components (viii), (ix) and (x) in a total concentration of from 1% to 10% by weight, based on a total weight of the preparation.

14. The sunscreen preparation of claim 3, wherein the preparation comprises components (x), (xi), (xii) and (xiii) in a total concentration of from 1% to 10% by weight, based on a total weight of the preparation.

15. The sunscreen preparation of claim 1, wherein a ratio of total weight of components (i), (ii), (iii) and (iv) to total weight of components (v), (vi) and (vii) is from 3:1 to 1:3.

16. The sunscreen preparation of claim 1, wherein a weight ratio of component (v) to component (vi) is from 6:1 to 2:1.

17. The sunscreen preparation of claim 1, wherein a weight ratio of component (v) to component (vii) is from 2:1 to 1:2.

18. The sunscreen preparation of claim 1, wherein the preparation has an in-vitro SPF of at least 30.

19. The sunscreen preparation of claim 1, wherein the preparation further comprises one or more of dibutyl adipate, silica dimethyl silylate, neopentyl glycol diheptanoate, butylene glycol dicaprylate/dicaprate, hydroxyacetophenone, silica.

20. A method of protecting skin from UV radiation, wherein the method comprises applying to the skin to be protected the sunscreen preparation of claim 1.
